(19) United States
(12) Patent Application Publication
(10) Pub. No.: US 2006/0281918 A1
(43) Pub. Date: Dec. 14, 2006

(54) Resolution of enantiomeric mixtures of beta-lactams

(75) Inventors: Phong Vu, Little Falls, NJ (US); Robert A. Holton, Tallahassee, FL (US)

Correspondence Address: SENNIGER POWERS, ONE METROPOLITAN SQUARE, 16TH FLOOR, ST LOUIS, MO, 63102, US

(73) Assignee: Florida State University Research Foundation, Inc., Tallahassee, FL (US)

(21) Appl. No.: 11/449,045

(22) Filed: Jun. 8, 2006

Related U.S. Application Data
(60) Provisional application No. 60/708,931, filed on Aug. 17, 2005.
(60) Provisional application No. 60/689,425, filed on Jun. 10, 2005.

(51) Int. Cl. C07D 205/08

(52) U.S. Cl. 540/354

(57) ABSTRACT

A new process for resolution of an enantiomeric mixture of C3-hydroxyl substituted β-lactams is disclosed. Generally, the enantiomeric mixture is treated with an optically active proline acylating agent to form a C3-ester substituted β-lactam diastereomer or a mixture of C3-ester substituted β-lactam diastereomers followed by selective recovery of the unreacted enantiomer or of one of the diastereomers.

### REFERENCE TO RELATED APPLICATIONS

This application claims the priority of U.S. provisional application Ser. No. 60/689,425, filed Jun. 10, 2005 and Ser. No. 60/708,931, filed Aug. 17, 2005, hereby incorporated herein by reference in their entirety.

### BACKGROUND

The present invention is generally directed to an improved process for the resolution of enantiomeric mixtures of β-lactams.

β-lactams possess biological activity and are used as synthetic intermediates for a variety of other biologically active compounds. Because the stereochemistry of these biologically active compounds may affect their pharmaceutical activity, methods allowing efficient stereospecific preparation of the β-lactam compounds have been the subject of investigation.

In U.S. Pat. No. 6,225,463, de Vos et al. describe reaction of a chiral imine with an acyl chloride to control the diastereoselectivity of the ring formation. In particular, the chiral imine is prepared from treatment of (S)-(−)-1-(p-methoxyphenyl)-propyl-1-amine with an aldehyde; (S)-(−)-1-(p-methoxyphenyl)-propyl-1-amine required an enantiomeric resolution for its preparation. This reaction produces a mixture of diastereomers that can be separated by crystallization.

In Synlett 1992, 9, 761-763, Farina et al. also describe reaction of a chiral imine with an acyl chloride for diastereo-control of the ring-forming step. In this instance, a 2-benzoxy- or 2-acetoxy-ethanoyl chloride was treated with an N-(L)-2-silylatedthreonine-2-phenyl imine, thus producing the corresponding cis-3-benzoxy or acetoxy-4-phenyl-azetidin-2-one (e.g., (3R,4S)- and (3S,4R)-) with diastereoselectivity as high as 19:1. But, it took a five-step reaction sequence to remove the (L)-threonine group attached to the nitrogen of the β-lactam.

Accordingly, a need exists for a process for preparing enantiomerically enriched β-lactams in fewer steps.

### SUMMARY

Among the various aspects of the present invention is an efficient process for preparing enantiomerically enriched β-lactams.

Another aspect is a process for the resolution of an enantiomeric mixture of first and second C3-hydroxy substituted β-lactam enantiomers comprising treating the enantiomeric mixture with an optically active proline acylating agent in the presence of an amine to form a product mixture. The product mixture contains first and second C3-ester substituted β-lactam diastereomers formed by reaction of the first and second C3-hydroxy substituted β-lactam enantiomers, respectively, with the optically active proline acylating agent. The product mixture optionally also containing unreacted second C3-hydroxy β-lactam enantiomer. The process also comprises separating the first C3-ester substituted β-lactam diastereomer from the unreacted second C3-hydroxy β-lactam enantiomer or the second C3-hydroxy substituted β-lactam diastereomer.

Yet another aspect of the present invention is a β-lactam compound having the structure of Formula 4 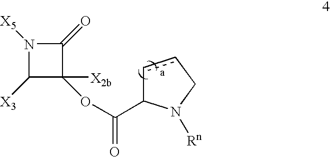
wherein

a is 1 or 2 whereby the heterocyclo ring is proline or homoproline;

the dashed line denotes an optional double bond between the C3 and C4 ring carbon atoms;

Rⁿ is a nitrogen protecting group;

X_{2b} is hydrogen, alkyl, alkenyl, alkynyl, aryl, heterocyclo, or —SX₇;

X₃ is alkyl, alkenyl, alkynyl, aryl, acyloxy, alkoxy, acyl or heterocyclo or together with X₅ and the carbon and nitrogen to which they are attached form heterocyclo;

X₅ is hydrogen, hydrocarbyl, substituted hydrocarbyl, —COX₁₀, —COOX₁₀, —CONX₈X₁₀, —SiR₅₁R₅₂R₅₃, or together with X₃ and the nitrogen and carbon to which they are attached form heterocyclo;

X₇ is hydrogen, hydrocarbyl, substituted hydrocarbyl, or heterocyclo;

X₈ is hydrogen, hydrocarbyl, substituted hydrocarbyl, or heterocyclo;

X₁₀ is hydrocarbyl, substituted hydrocarbyl, or heterocyclo; and

R₅₁, R₅₂, and R₅₃ are independently alkyl, aryl or aralkyl.

Other objects and features will be in part apparent and in part pointed out hereinafter.

### DETAILED DESCRIPTION

In accordance with the present invention, a process has been discovered which enables the resolution of an enantiomeric mixture of a C3-hydroxy substituted β-lactam using commercially available, optically enriched proline. Advantageously, this approach results in a β-lactam having a high enantiomeric excess and the process has fewer steps than conventional processes.

Because enantiomers have identical physical properties such as solubility, but rotate polarized light in opposite directions, they are difficult to separate by standard physical and chemical methods. When C3-hydroxy substituted β-lactam enantiomers are placed in a chiral environment, however, their properties are distinguishable. One way to place the enantiomers in a chiral environment is to react them with an optically active proline acylating agent to produce C3-ester substituted diastereomers. Depending on the extent of reaction from reactants (e.g., C3-hydroxy substituted enantiomers) to product(s) (e.g., C3-ester substituted diastereomer(s)), either (1) the differential reactivity of the enantiomers with the optically active proline acylating agent (i.e., kinetic resolution) or (2) the conversion of the enantiomers to diastereomers by reaction with the optically active proline acylating agent (i.e., classical resolution) is used to chemically and physically distinguish the enantiomers. In the method exploiting the differential reactivity of the enantiomers with the optically active proline acylating agent, the reaction conditions are changed to maximize the conversion of the more reactive C3-hydroxy substituted β-lactam enantiomer (or first C3-hydroxy substituted β-lactam enantiomer) to the corresponding diastereomer, while minimizing the conversion of the less reactive C3-hydroxy substituted β-lactam enantiomer (or second C3-hydroxy substituted β-lactam enantiomer) to the corresponding diastereomer. For example, as the more reactive enantiomer reacts with the optically active proline acylating agent, the concentration of the more reactive enantiomer becomes depleted and its rate of conversion to the corresponding diastereomer slows. Concurrently, the rate of the reaction of the optically active proline acylating agent with the less reactive enantiomer increases.

Depending on, for example, the time, temperature, and starting material ratios, the reaction can be controlled so that varying amounts of the less reactive enantiomer reacts with the optically active proline acylating agent to form a diastereomer. For example, timing the reaction progress to end the reaction when the more reactive enantiomer is substantially reacted, but the less reactive enantiomer is substantially unreacted, lowering the temperature of the reaction to enhance the reaction rate difference between the enantiomers, and reducing the ratio of the optically active proline acylating agent to the enantiomeric mixture (e.g., 0.5:1) favor the production of the diastereomer corresponding to the more reactive enantiomer over the production of the diastereomer corresponding to the less reactive enantiomer.

The more reactive enantiomer is substantially reacted, for example, when at least about 70%, preferably at least about 80%, more preferably at least about 90% (on a weight or mole basis) of the enantiomer reacts with the optically active proline acylating agent to form a C3-ester substituted diastereomer. Similarly, the less reactive enantiomer is substantially unreacted, for example, when less than about 30%, preferably, less than about 20%, more preferably, less than about 10% (on a weight or mole basis) of the enantiomer reacts with the optically active proline acylating agent.

Alternatively, the reaction time, reaction temperature and the starting material ratios can be adjusted to favor substantially complete conversion of the C3-hydroxy substituted β-lactam enantiomers to the corresponding C3-ester substituted β-lactam diastereomers. For example, when the reaction time is longer, the reaction temperature is higher, and the ratio of the optically active proline acylating agent to enantiomer is higher (e.g., 1:1), the complete conversion to diastereomers is favored. These diastereomers can then be chemically or physically separated from each other to produce the desired enantiomer upon hydrolysis of the corresponding diastereomer.

Further, the enantiomeric excess of the optically active proline acylating agent is important. The higher the enantiomeric excess, the higher the concentration of one pair of the two possible pairs of diastereomers. By forming substantially one or one pair of diastereomers depending on whether it is a kinetic or classical resolution, the separation of the products formed is facilitated. Thus, use of an optically active proline acylating agent having lower enantiomeric excesses is possible, but preferably, the optically active proline acylating agent has an enantiomeric excess of at least about 70% e.e.

In the kinetic resolution process, D-proline preferentially reacts with one member of the enantiomeric pair to form an ester derivative whereas L-proline preferentially reacts with the other member of the enantiomeric pair to form an ester derivative. Thus, a racemic or other enantiomeric mixture of C3-hydroxy substituted β-lactam enantiomers can be optically enriched in one of the enantiomers by (i) treating the original mixture with enantiomerically enriched D-proline or L-proline to preferentially convert one of the β-lactam enantiomers to an ester derivative and (ii) separating the unreacted enantiomer from the ester derivative.

One embodiment of the kinetic resolution method of the present invention is illustrated in Scheme 1. In this embodiment, an enantiomeric mixture of C3-hydroxy substituted β-lactams, cis-1 and cis-2, is treated with an optically active L-proline acylating agent 3L and an amine to form a C3-ester substituted β-lactam diastereomer cis-4. Preferably, the optically active proline acylating agent has at least about a 70% enantiomeric excess (“e.e.”), that is, 85 weight or mole percent of one enantiomer and 15 weight or mole percent of the other enantiomer. More preferably, the optically active proline acylating agent has at least about a 90% enantiomeric excess. Still more preferably, the optically active proline has at least about a 95% enantiomeric excess. In one particularly preferred embodiment, the optically active proline has at least about a 98% enantiomeric excess. Scheme 1 follows 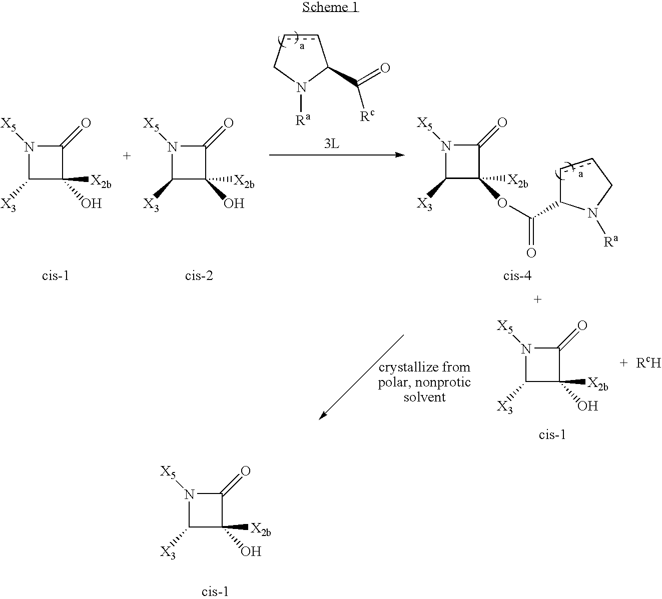
wherein

a is 1 or 2 whereby the heterocyclo ring is proline or homoproline;

the dashed line denotes an optional double bond between the C3 and C4 ring carbon atoms;

R^{c} is hydroxy, amino, halo, —OC(O)R₃₀;

Rⁿ is nitrogen protecting group;

R₃₀ is hydrocarbyl, substituted hydrocarbyl or heterocyclo;

X_{2b} is hydrogen, alkyl, alkenyl, alkynyl, aryl, heterocyclo, or —SX₇;

X₃ is alkyl, alkenyl, alkynyl, aryl, acyloxy, alkoxy, acyl or heterocyclo or together with X₅ and the carbon and nitrogen to which they are attached form heterocyclo;

X₅ is hydrogen, hydrocarbyl, substituted hydrocarbyl, —COX₁₀, —COOX₁₀, —CONX₈X₁₀, —SiR₅₁R₅₂R₅₃, or together with X₃ and the nitrogen and carbon to which they are attached form heterocyclo;

X₇ is hydrogen, hydrocarbyl, substituted hydrocarbyl, or heterocyclo;

X₈ is hydrogen, hydrocarbyl, substituted hydrocarbyl, or heterocyclo;

X₁₀ is hydrocarbyl, substituted hydrocarbyl, or heterocyclo; and

R₅₁, R₅₂, and R₅₃ are independently alkyl, aryl or aralkyl.

An alternative embodiment of the kinetic resolution method of the present invention is illustrated in Scheme 2. In this embodiment, the enantiomeric mixture of C3-hydroxy substituted β-lactams, cis-1 and cis-2, is treated with an amine and an optically active proline acylating agent 3 having an enantiomeric excess of enantiomer 3D to form a C3-ester substituted β-lactam diastereomer cis-5. Scheme 2 follows 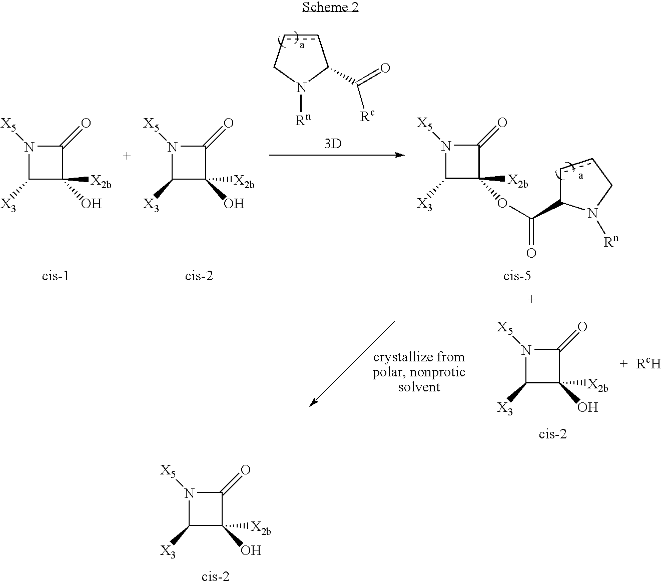
wherein a, the dashed line, R^{c}, Rⁿ, X_{2b}, X₃, X₅, X₇, X₈ and X₁₀ are as defined in connection with Scheme 1.

By controlling the enantiomeric purity of the proline reactant in Schemes 1 and 2, therefore, diastereomer cis-4 or diastereomer cis-5 is preferentially formed. Because diastereomer cis-4 and enantiomer cis-1 (Scheme 1) have different physical properties, enantiomer cis-1 can be readily crystallized from a polar, nonprotic solvent. Similarly, because diastereomer cis-5 and enantiomer cis-2 (Scheme 2) have different physical properties, enantiomer cis-2 can be readily crystallized from a polar, nonprotic solvent.

In the classical resolution process, the proline acylating agent reacts with both members of the enantiomeric pair to form ester derivatives that are a diastereomeric pair. Thus, a racemic or other enantiomeric mixture of C3-hydroxy substituted β-lactam enantiomers can be optically enriched in one of the enantiomers by (i) treating the original mixture with enantiomerically enriched D-proline or L-proline acylating agent to convert each of the β-lactam enantiomers to ester derivatives thus forming a diastereomeric mixture and (ii) separating the physically distinguishable β-lactam diastereomers from each other.

One embodiment of the classical resolution method of the present invention is illustrated in Scheme 1A. In this embodiment, an enantiomeric mixture of C3-hydroxy substituted β-lactams, cis-1 and cis-2, is treated with an optically active L-proline acylating agent 3L to form C3-ester substituted β-lactam diastereomers cis-4 and cis-4A. Scheme 1A follows 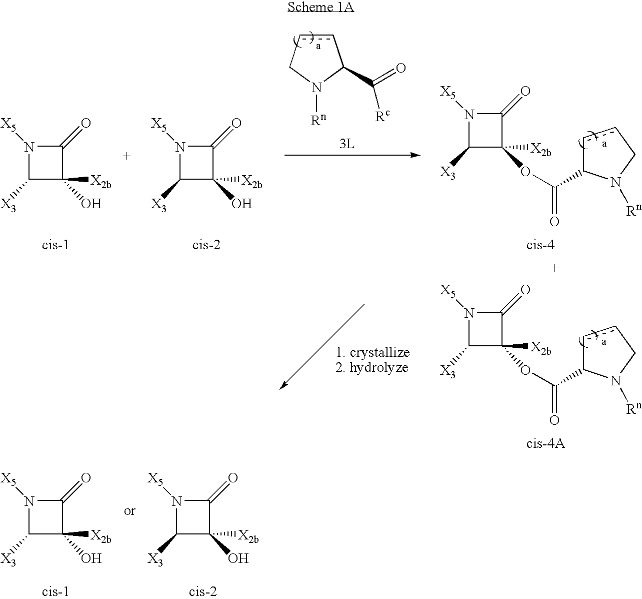
wherein a, the dashed line, R^{c}, Rⁿ, X_{2b}, X₃, X₅, X₇, X₈ and X₁₀ are as defined in connection with Scheme 1.

Alternately, another embodiment of the classical resolution method is illustrated in Scheme 2A. In this embodiment, an enantiomeric mixture of C3-hydroxy substituted β-lactams, cis-1 and cis-2, is treated with an optically active D-proline acylating agent 3D to form C3-ester substituted β-lactam diastereomers cis-5 and cis-5A. Scheme 2A follows 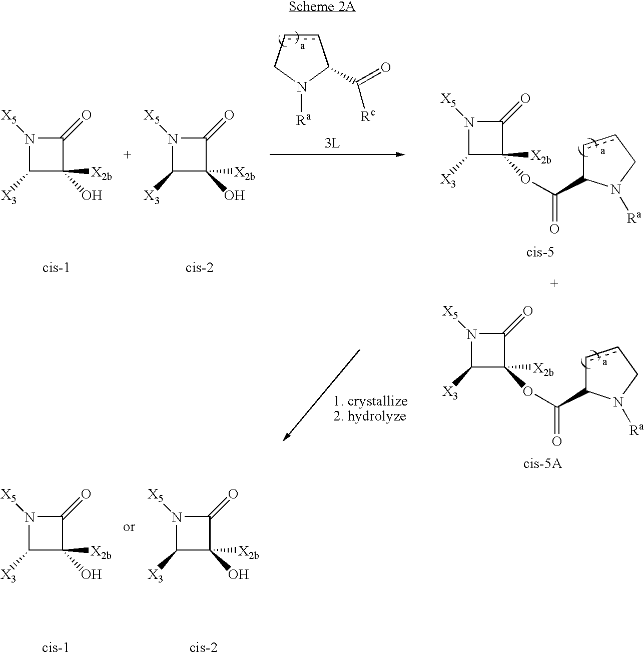
wherein a, the dashed line, R^{c}, Rⁿ, X_{2b}, X₃, X₅, X₇, X₈ and X₁₀ are as defined in connection with Scheme 1. The reagents are chosen to produce the desired stereochemistry for the particular synthetic or biological application of the enantiomerically enriched β-lactam products.
Enantiomerically Enriched β-lactams

Because enantiomer cis-1 or a diastereomer of cis-1 can be crystallized from the reaction mixture as described above, one aspect of the present invention is a process for enantiomeric enrichment of a β-lactam corresponding to 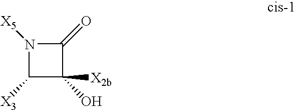
wherein X_{2b}, X₃, and X₅ are as defined in connection with Scheme 1.

Similarly, because enantiomer cis-2 or a diastereomer of cis-2 can be crystallized from the reaction mixture as described above, another aspect of the present invention is a process for enantiomeric enrichment of a β-lactam corresponding to Formula 2 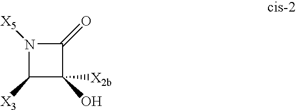
wherein X_{2b}, X₃, and X₅, are as defined in connection with Scheme 1.

Although X_{2b} may be hydrogen, alkyl, alkenyl, alkynyl, aryl or heterocyclo, in one embodiment, X_{2b} is hydrogen, alkyl or aryl. In one preferred embodiment, X_{2b} is hydrogen.

Similarly, although X₃ may be alkyl, alkenyl, alkynyl, aryl, acyloxy, alkoxy, acyl or heterocyclo, or together with X₅ and the carbon and nitrogen to which they are attached form heterocyclo, in one embodiment, X₃ is alkyl, aryl or heterocyclo. For example, X₃ may be phenyl. In another embodiment, X₃ is furyl or thienyl. In yet another embodiment, X₃ is cycloalkyl.

As previously noted, X₅ may be hydrogen, hydrocarbyl, substituted hydrocarbyl, —COX₁₀, —COOX₁₀, —CONX₈X₁₀ or together with X₃ and the nitrogen and carbon to which they are attached form heterocyclo. For example, in one embodiment, X₅ is hydrogen. In an alternative embodiment, X₅ is —COX₁₀ and X₁₀ is alkyl, alkenyl or aryl; for example, X₅ may be —COX₁₀ and X₁₀ is phenyl. In another alternative embodiment, X₅ is —COOX₁₀ and X₁₀ is alkyl; for example, X₅ may be —COOX₁₀ and X₁₀ is n-propyl, isopropyl, n-butyl, isobutyl or tert-butyl. In yet another alternative embodiment, X₅ is —COOX₁₀ and X₁₀ is tert-butyl.

In combination, among the preferred embodiments are β-lactams corresponding to Formula 1 wherein X_{2b} is hydrogen; X₃ is alkyl, aryl or heterocyclo, preferably, cycloalkyl, more preferably, phenyl, furyl or thienyl; and X₅ is hydrogen, alkylcarbonyl, alkenylcarbonyl, aroyl or alkoxycarbonyl, preferably, benzoyl, alkoxycarbonyl, more preferably, benzoyl, n-propoxycarbonyl, isopropoxycarbonyl, isobutoxycarbonyl or tert-butoxycarbonyl.

Diastereomeric Mixtures of β-lactams

As described above in Scheme 1, in a kinetic resolution process a β-lactam diastereomer cis-4 is prepared and in a classical resolution process (see Scheme 1A) a mixture of β-lactam diastereomers (cis-4 and cis-4A) are prepared. Structures corresponding to Formulae cis-4 and cis-4A follow 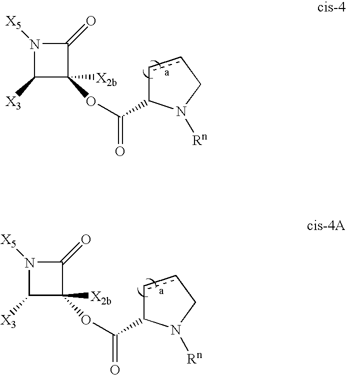
wherein a, the dashed line, Rⁿ, X_{2b}, X₃, X₅, X₇, X₈ and X₁₀ are as defined above in connection with Scheme 1.

As described above in Scheme 2, in a kinetic resolution process a β-lactam diastereomer cis-5 is prepared and in a classical resolution process (see Scheme 2A) a mixture of β-lactam diastereomers (cis-5 and cis-5A) are prepared. Structures corresponding to Formulae cis-5 and cis-5A follow 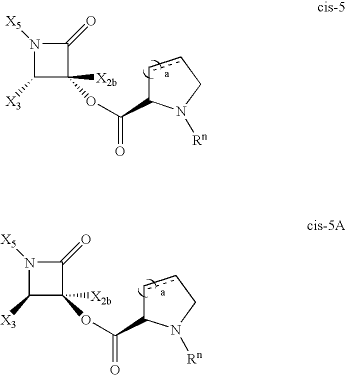
wherein Rⁿ, X_{2b}, X₃, X₅, X₇, X₈ and X₁₀ are as defined in connection with Scheme 1.

In one embodiment, Rⁿ is t-butoxycarbonyl or carbobenzyloxy. Preferred substituent groups for X_{2b}, X₃, X₅ and X₁₀ are detailed above for Formula cis-1.

Among the preferred embodiments are β-lactams corresponding to Formula cis-4 and cis-4A wherein Rⁿ is t-butoxycarbonyl or carbobenzyloxy; X_{2b} is hydrogen; X₃ is alkyl, aryl or heterocyclo, preferably, cycloalkyl, more preferably, phenyl, furyl or thienyl; and X₅ is hydrogen, alkylcarbonyl, alkenylcarbonyl, aroyl or alkoxycarbonyl, preferably, benzoyl, alkoxycarbonyl, more preferably, benzoyl, n-propoxycarbonyl, isopropoxycarbonyl, isobutoxycarbonyl or tert-butoxycarbonyl.

In other embodiments are β-lactams corresponding to Formula cis-5 and cis-5A wherein Rⁿ is t-butoxycarbonyl or carbobenzyloxy and X_{2b} is hydrogen. In these embodiments, X₃ is alkyl, aryl or heterocyclo, preferably, cycloalkyl, more preferably, phenyl, furyl or thienyl; and X₅ is hydrogen, alkylcarbonyl, alkenylcarbonyl, aroyl or alkoxycarbonyl, preferably, benzoyl, alkoxycarbonyl, more preferably, benzoyl, n-propoxycarbonyl, isopropoxycarbonyl, isobutoxycarbonyl or tert-butoxycarbonyl.

Diastereomers cis-4, cis-4A, cis-5, and cis-5A are prepared by reacting each enantiomer with an optically enriched proline acylating agent 3 as described in more detail below.

Enantiomeric Mixtures of β-lactams

In one aspect of the present invention, the process is used to separate an enantiomeric mixture of β-lactams cis-1 and cis-2 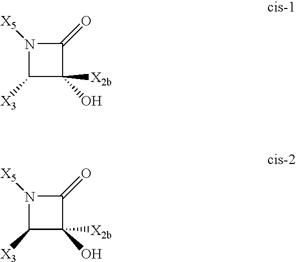
wherein X_{2b}, X₃, X₅, X₇, X₈ and X₁₀ are defined above in connection with Scheme 1.

Preferred substituent groups are defined as above for Formula cis-1.

Generally, the enantiomeric mixtures of β-lactams can be prepared by treatment of an imine with an acyl chloride or lithium enolate as described in U.S. Pat. No. 5,723,634 herein incorporated by reference. Further, the enantiomeric mixtures of β-lactams can be prepared from treatment of an imine with a (thio)ketene acetal or enolate in the presence of an alkoxide or siloxide as described below. A preferred embodiment of this cyclocondensation reaction is illustrated in Reaction Scheme 3 in which imine 12 is cyclocondensed with ketene (thio)acetal or enolate 13 to produce β-lactam 11. 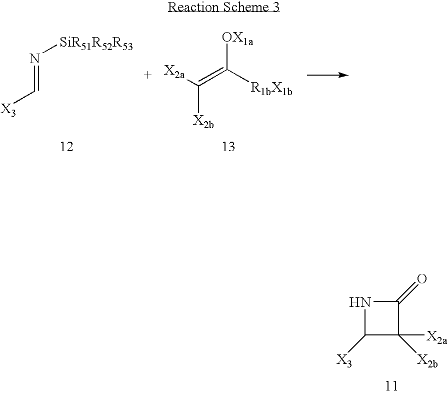
The ketene acetal is commercially available or may be prepared in situ from a carboxylic acid and the enolate can be prepared in situ from a carboxylic acid. The imine may be prepared in situ from commercially available aldehydes and disilazides. With respect to Reaction Scheme 3, X₁ₐ a silyl protecting group, metal, or comprises ammonium; X_{1b} is a sulfhydryl or hydroxyl protecting group; X₂ₐ is hydrogen, alkyl, alkenyl, alkynyl, aryl, heterocyclo, —OX₆, —SX₇, or —NX₈X₉; X_{2b} is hydrogen, alkyl, alkenyl, alkynyl, aryl, heterocyclo, —OX₆, or —SX₇; X₃ is alkyl, alkenyl, alkynyl, aryl or heterocyclo; X₆ is alkyl, alkenyl, alkynyl, aryl, heterocyclo, or hydroxyl protecting group; X₇ is alkyl, alkenyl, alkynyl, aryl, heterocyclo, or sulfhydryl protecting group; X₈ is hydrogen, hydrocarbyl, substituted hydrocarbyl, or heterocyclo; X₉ is hydrogen, amino protecting group, hydrocarbyl, substituted hydrocarbyl, or heterocyclo; R_{1b} is oxygen or sulfur; and R₅₁, R₅₂ and R₅₃ are independently alkyl, aryl, or aralkyl.
Optically Active Proline or Proline Derivative

Preferably, the proline acylating agent corresponds to Formula 3 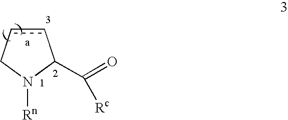
wherein a, the dashed line, R^{c} and Rⁿ are defined above in connection with Scheme 1. Where R^{c} is hydroxy, a proline acylating agent is prepared by treating the proline free acid with an acid acylating agent.

In preferred embodiments, a is 1, there is not a double bond between the C3 and C4 ring carbon atoms, R^{c} is hydroxyl, and Rⁿ is t-butoxycarbonyl or carbobenzyloxy.

In many of the various embodiments, the optically active proline acylating agent has at least about a 70% enantiomeric excess (e.e.); in a further embodiment, at least about a 90% e.e.; preferably, at least about a 95% e.e.; more preferably, at least about a 98% e.e.

Treatment of Enantiomeric Mixtures of β-lactams with Optically Active Proline or Proline Derivative

As depicted above in Schemes 1 and 2, in the kinetic resolution method, when an enantiomeric mixture of β-lactams (cis-1 and cis-2) is treated with an optically active proline acylating agent 3 and an amine, a diastereomer is formed (cis-4 or cis-5). The optically active proline or proline derivative used as the proline acylating agent can be a free acid, an acid halide, an anhydride, or a mixed anhydride. When the optically active proline or proline derivative is in the free acid form, treatment of the free acid with an acid acylating agent to form an optically active proline acylating agent is necessary for the product to be obtained. But, when the optically active proline or proline derivative is in the acid halide, anhydride, or mixed anhydride form, reaction with the acid acylating agent is not necessary because these forms of the proline are optically active proline acylating agents.

Further, the reaction of the enantiomeric mixture of β-lactams (cis-1 and cis-2) to form a diastereomer (cis-4 or cis-5) or a diastereomeric mixture (cis-4 and cis-5) requires an amine. Preferred amine bases are aromatic amine bases such as substituted or unsubstituted pyridines (e.g., pyridine, N,N′-dimethylaminopyridine (DMAP)), or substituted or unsubstituted imidazoles (e.g., imidazole, 1-methylimidazole, 1,2-dimethylimidazole, benzimidazole, N,N′-carbonyldiimidazole), and the like.

Exemplary acid acylating agents for conversion of proline free acids to proline acylating agents are p-toluenesulfonyl chloride (TsCl), methanesulfonyl chloride (MsCl), oxalic acid chloride, di-t-butyl dicarbonate (Boc₂O), dicyclohexylcarbodiimide (DCC), alkyl chloroformate, 2-chloro-1,3,5-trinitrobenzene, polyphosphate ester, chlorosulfonyl isocyanate, Ph₃P—CCl₄, and the like. Preferably, the acid acylating agent is p-toluenesulfonyl chloride (TsCl), methanesulfonyl chloride (MsCl), oxalic acid chloride, or di-t-butyl dicarbonate (Boc₂O). In various embodiments, the acid acylating agent is p-toluenesulfonyl chloride or methanesulfonyl chloride.

In one embodiment of the present invention an enantiomeric mixture of β-lactams (cis-1 and cis-2) is treated with an L-proline acylating agent in the presence of an amine to form a β-lactam diastereomer (cis-4). Preferably, the enantiomeric mixture is treated with L-proline in the presence of an acid acylating agent (e.g., p-toluenesulfonyl chloride) and an amine.

Specifically, when treating an enantiomeric mixture of cis-1 and cis-2 with L-proline in the presence of an amine and less than a stoichiometrically equivalent amount of p-toluenesulfonyl chloride resulted in diastereomer cis-4. For cis-4, when X_{2b} is hydrogen, X₃ is furyl and X₅ is hydrogen, desired cis-1 preferentially crystallizes and recrystallization from ethyl acetate can provide the desired β-lactam product in high enantiomeric excess (e.g., 98% e.e. or more).

The enantiomer (cis-2 or cis-1) can be separated from the diastereomer (cis-4 or cis-5) by physical methods known in the art. For example, they can be separated by crystallization, liquid chromatography and the like.

Once the desired enantiomer is crystallized, the remaining diastereomer (e.g., cis-4) can be reacted with an aqueous base or aqueous acid to form the corresponding C3-hydroxyl β-lactam.

Alternatively, in the classical resolution method, an enantiomeric mixture of cis-1 and cis-2 can be treated with an L-proline acylating agent in the presence of an amine to result in diastereomers cis-4 and cis-4A. Where X_{2b} is hydrogen, X₃ is phenyl and X₅ is hydrogen, upon dissolution of a portion of the diastereomeric mixture in warm (40° C.) ethyl acetate, the desired 3R,4S-diastereomer (cis-4A) crystallized from solution. When the filtrate was allowed to stand at room temperature for several hours, the 3S,4R-diasteromer (cis-4) crystallized from the solution. Removal of the proline ester of cis-4 or cis-4A to form the optically enriched C3-hydroxyl β-lactams cis-1 and cis-2 can be accomplished by hydrolysis of the ester moiety. When a D-proline acylating agent is used in this process, diastereomers cis-5 and cis-5A are formed and optically enriched C3-hydroxyl β-lactams cis-1 and cis-2 can be obtained using a similar process.

Definitions

The term “acyl,” as used herein alone or as part of another group, denotes the moiety formed by removal of the hydroxyl group from the group —COOH of an organic carboxylic acid, e.g., RC(O)—, wherein R is R¹, R¹O—, R¹R²N—, or R¹S—, R¹ is hydrocarbyl, heterosubstituted hydrocarbyl, or heterocyclo, and R² is hydrogen, hydrocarbyl or substituted hydrocarbyl.

The term “acyloxy,” as used herein alone or as part of another group, denotes an acyl group as described above bonded through an oxygen linkage (—O—), e.g., RC(O)O— wherein R is as defined in connection with the term “acyl.”

Unless otherwise indicated, the alkyl groups described herein are preferably lower alkyl containing from one to eight carbon atoms in the principal chain and up to 20 carbon atoms. They may be substituted or unsubstituted and straight or branched chain or cyclic and include methyl, ethyl, propyl, butyl, pentyl, hexyl and the like. The substituted alkyl groups can be substituted with, for example, aryl, amino, hydroxyl, imino, amido, carboxyl, thio, mercapto and heterocyclo.

Unless otherwise indicated, the alkenyl groups described herein are preferably lower alkenyl containing from two to eight carbon atoms in the principal chain and up to 20 carbon atoms. They may be substituted or unsubstituted and straight or branched chain or cyclic and include ethenyl, propenyl, butenyl, pentenyl, hexenyl, and the like.

Unless otherwise indicated, the alkynyl groups described herein are preferably lower alkynyl containing from two to eight carbon atoms in the principal chain and up to 20 carbon atoms. They may be substituted or unsubstituted and straight or branched chain and include ethynyl, propynyl, butynyl, pentynyl, hexynyl, and the like.

The “amino protecting groups” described herein are moieties that block reaction at the protected amino group while being easily removed under conditions that are sufficiently mild so as not to disturb other substituents of the various compounds. For example, the amino protecting groups may be carbobenzyloxy (Cbz), t-butoxycarbonyl (t-Boc), allyloxycarbonyl and the like. A variety of protecting groups for the amino group and the synthesis thereof may be found in “Protective Groups in Organic Synthesis” by T. W. Greene and P. G. M. Wuts, John Wiley & Sons, 1999.

The term “aromatic” as used herein alone or as part of another group denote optionally substituted homo- or heterocyclic aromatic groups. These aromatic groups are preferably monocyclic, bicyclic, or tricyclic groups containing from 6 to 14 atoms in the ring portion. The term “aromatic” encompasses the “aryl” and “heteroaryl” groups defined below.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The term “aralkyl” as used herein denote optionally substituted alkyl groups substituted with an aryl group. Exemplary aralkyl groups are substituted or unsubstituted benzyl, ethylphenyl, propylphenyl and the like.

The term “carboxylic acid” refers to a RC(O)OH compound where R can be hydrogen, or substituted or unsubstituted alkyl, alkenyl, alkynyl, aryl, substituted aryl.

The term “heteroatom” shall mean atoms other than carbon and hydrogen.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms and/or 1 to 4 nitrogen atoms in the ring, and is bonded to the remainder of the molecule through a carbon or heteroatom. Exemplary heterocyclo groups include tetrahydrofuryl, tetrahydropyrrolyl, tetrahydropyranyl and heteroaromatics as described below. Exemplary substituents include one or more of the following groups: hydrocarbyl, substituted hydrocarbyl, hydroxy, protected hydroxy, acyl, acyloxy, alkoxy, alkenoxy, alkynoxy, aryloxy, halogen, amido, amino, cyano, ketals, acetals, esters and ethers.

The term “heteroaryl” as used herein alone or as part of another group denote optionally substituted aromatic groups having at least one heteroatom in at least one ring, and preferably 5 or 6 atoms in each ring. The heteroaryl group preferably has 1 or 2 oxygen atoms and/or 1 to 4 nitrogen atoms and/or 1 or 2 sulfur atoms in the ring, and is bonded to the remainder of the molecule through a carbon. Exemplary heteroaryls include furyl, thienyl, pyridyl, oxazolyl, isoxazolyl, oxadiazolyl, pyrrolyl, pyrazolyl, triazolyl, tetrazolyl, imidazolyl, pyrazinyl, pyrimidyl, pyridazinyl, thiazolyl, thiadiazolyl, biphenyl, naphthyl, indolyl, isoindolyl, indazolyl, quinolinyl, isoquinolinyl, benzimidazolyl, benzotriazolyl, imidazopyridinyl, benzothiazolyl, benzothiadiazolyl, benzoxazolyl, benzoxadiazolyl, benzothienyl, benzofuryl and the like. Exemplary substituents include one or more of the following groups: hydrocarbyl, substituted hydrocarbyl, hydroxy, protected hydroxy, acyl, acyloxy, alkoxy, alkenoxy, alkynoxy, aryloxy, halogen, amido, amino, cyano, ketals, acetals, esters and ethers.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxy, alkynoxy, aryloxy, hydroxy, protected hydroxy, acyl, acyloxy, nitro, amino, amido, nitro, cyano, ketals, acetals, esters and ethers.

The “hydroxyl protecting groups” described herein are moieties that block reaction at the protected hydroxyl group while being easily removed under conditions that are sufficiently mild so as not to disturb other substituents of the various compounds. For example, the hydroxyl protecting groups may be ethers (e.g., allyl, triphenylmethyl (trityl or Tr), benzyl, p-methoxybenzyl (PMB), p-methoxyphenyl (PMP)), acetals (e.g., methoxymethyl (MOM), β-methoxyethoxymethyl (MEM), tetrahydropyranyl (THP), ethoxy ethyl (EE), methylthiomethyl (MTM), 2-methoxy-2-propyl (MOP), 2-trimethylsilylethoxymethyl (SEM)), esters (e.g., benzoate (Bz), allyl carbonate, 2,2,2-trichloroethyl carbonate (Troc), 2-trimethylsilylethyl carbonate), silyl ethers (e.g., trimethylsilyl (TMS), triethylsilyl (TES), triisopropylsilyl (TIPS), triphenylsilyl (TPS), t-butyldimethylsilyl (TBDMS), t-butyldiphenylsilyl (TBDPS) and the like. A variety of protecting groups for the hydroxyl group and the synthesis thereof may be found in “Protective Groups in Organic Synthesis” by T. W. Greene and P. G. M. Wuts, John Wiley & Sons, 1999.

The “sulfhydryl protecting groups” described herein are moieties that block reaction at the protected sulfhydryl group while being easily removed under conditions that are sufficiently mild so as not to disturb other substituents of the various compounds. For example, the sulfhydryl protecting groups may be silyl esters, disulfides and the like. More particularly, thiol protecting groups of triphenylmethyl, acetamidomethyl, benzamidomethyl, and 1-ethoxyethyl, benzoyl and protected thiol groups of alkylthio, acylthio, thioacetal, aralkylthio (e.g., methylthio, ethylthio, propylthio, isopropylthio, butylthio, isobutylthio, sec-butylthio, tert-butylthio, pentylthio, isopentylthio, neopentylthio, hexylthio, heptylthio, nonylthio, cyclobutylthio, cyclopentylthio and cyclohexylthio, benzylthio, phenethylthio, propionylthio, n-butyrylthio, and iso-butyrylthio). A variety of protecting groups for the sulfhydryl group and the synthesis thereof may be found in “Protective Groups in Organic Synthesis” by T. W. Greene and P. G. M. Wuts, John Wiley & Sons, 1999.

The following examples illustrate the invention.

### EXAMPLES

### Example 1

### Resolution of (±)-Cis-3-hydroxy-4-(2-furyl)-azetidin-2-one

(±)-—Cis-3-hydroxy-4-(2-furyl)-azetidin-2-one (500 g, 3.265 mol) was treated with N-t-Boc-L-proline (378.83 g, 1.76 mol) in the presence of 0.5 equivalents of p-toluenesulfonyl chloride (335.53 g, 1.76 mol) and 1-methyl-imidazole (303.45 g, 3.7 mol) at −78° C. for 12 hours. The mixture was filtered through 5 kg of silica gel. The undesired (−)-β-lactam enantiomer of t-Boc-L-proline ester was removed by trituration with water. The desired enantiomer was recovered by azeotropic removal of the water with 2-methyl-1-propanol and recrystallized from ethyl acetate to give the desired (+)-cis-3-hydroxy-4-(2-furyl)-azetidin-2-one. The optical purity after recrystallizing from ethyl acetate was greater than 98%. mp: 133 to 135° C.; [α]²⁰ D=+109.5 (MeOH, c=1.0), ¹H NMR (400 MHz, CDCl₃) (ppm): 2.69 (bs, 1H), 4.91 (d, J=4.96 Hz, 1H), 5.12 (bs, 1H), 6.10 (bs, 1H), 6.34 (dd, J=3.32, 3.32 Hz, 1H), 6.47 (d, J=3.32 Hz, 1H), 7.49 (m, 1H).

### Example 2

### Resolution of (±)-Cis-3-hydroxy-4-phenyl-azetidin-2-one

(±)-Cis-3-hydroxy-4-phenyl-azetidin-2-one (60 g, 0.368 mol) was treated with N-cBz-L-proline (45 g, 0.184 mol) in the presence of 0.5 equivalents of p-toluenesulfonyl chloride (35 g, 0.184 mol) and 1-methylimidazole (45 mL, 0.56 mol) at −78° C. for 12 hours. After concentration of the reaction mixture and filtration through silica gel to remove the 1-methylimidazolium tosylate salt, the desired diastereomer was crystallized from ethyl acetate to give 14.5 g (48%) of a white solid. This protocol resulted in kinetic resolution of the enantiomeric mixture to give the desired (+)-cis-3-hydroxy-4-phenyl-azetidin-2-one. The optical purity after recrystallizing from ethyl acetate was greater than 98%. mp: 175 to 180° C.; [α]₅₇₈²⁰=+202 (MeOH, c=1.0), ¹H NMR (400 MHz, CDCl₃) (ppm): 2.26 (d, J=9.4 Hz, 1H), 4.96 (d, J=4.96 Hz, 1H), 5.12 (m, 1H), 4.15 (bm, 1H), 7.41 (m, 5H).

### Example 3

### Kinetic Resolution of (±)-Cis-3-hydroxy-4-phenyl-azetidin-2-one

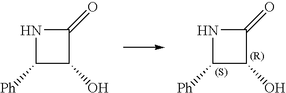

To a dry 250-mL round bottom flask under nitrogen was added acetonitrile (50 mL) and 1-methyl-imidazole (28 g, 0.2 mol) and the mixture was cooled to 0 to 5° C. Methanesulfonyl chloride (MsCl, 17.44 g, 0.1 mol) was added slowly to the mixture to control the exothermic reaction. After the reaction temperature was cooled to 0 to −5° C., N-cBz-L-proline (25 g, 0.1 mol) was added and the mixture was stirred at this temperature for 30 min. In a separate 3-L flask under nitrogen, racemic (±)-cis-3-hydroxy-4-phenyl-azetidin-2-one (16.3 g, 0.1 mol) was dissolved in acetone (1 L) and cooled to −65 to −78° C. and stirred mechanically. Once the temperature reached below −65° C., the content of the flask containing the proline reagent was added to the acetone solution of the racemic starting material. The mixture was kept at this temperature for a minimum of 6 h and a white precipitate was observed. The precipitate was allowed to settle and supernatant was transferred to the rotary evaporator as a cold solution (circa −45° C.) via vacuum suction through an immersion filter. The acetone was removed and exchanged with ethyl acetate (500 mL) and triethylamine (50 g, 5 eq) base. The resulting salt was filtered off and the filtrate was concentrated to approximately 100 mL and crystal formation was allowed to occur. The crystals were collected via vacuum filtration through a Buchner funnel, washed with cold ethyl acetate, and dried under vacuum (0.1 mmHg) at ambient temperature to a constant weight of 7.5 g (46%).

The efficiency of the kinetic resolution was determined by the ratio of the diastereomeric ester (SSS:RRS) of the beta-lactam with the Boc-L-proline via ¹HNMR according to Scheme 4. In the table TsCl is tosyl chloride, Boc₂O is di-tert-butyidicarbonate, MsCl is mesyl chloride and MstCl is mesityl chloride.

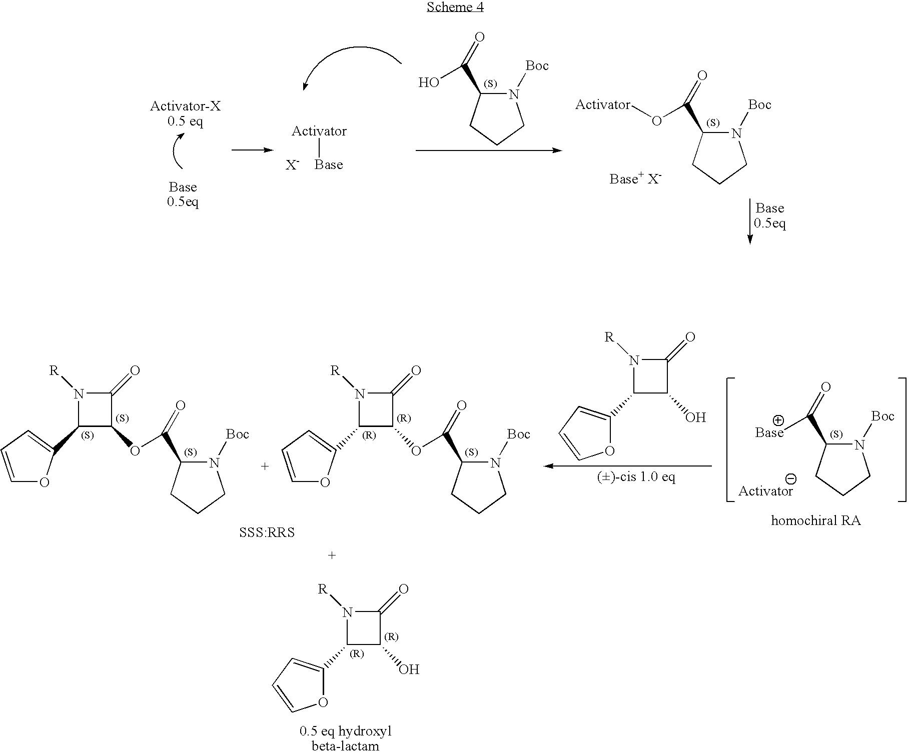

| | | | | Temp | | Time h/ | Dr |
|---|---|---|---|---|---|---|---|
| Entry | R | Activator | Base | (° C.) | Solvent | % Conv. | SSS:RRS |
| 1 | PMP | TsCl | 1-methyl-imidazole | −78 | DME/ACN | 3/50 | 10:1 |
| 2 | H | TsCl | 1-methyl-imidazole | −78 | DME/ACN | 3/50 | 8.5:1 |
| 3 | H | TsCl | 1-methyl-imidazole | 0 | ACN | 3/50 | 2.6:1 |
| 4 | H | TsCl | triethylamine | 0 | ACN | 3/15 | 1:2.9 |
| 5 | H | TsCl | 1- | −78 to | DME/ACN | 12/50 | 8:1 |
| | | | methylbenzimidazole | 22 | | | |
| 6 | H | TsCl | 1,2- | −78 | DME/ACN | 3/50 | 4.5:1 |
| | | | dimethylimidazole | | | | |
| 7 | H | TsCl | Pyridine | −40 | Pyridine | 6/20 | 6.8:1 |
| 8 | H | TsCl | Pyridine | 0 | Pyridine | 3/50 | 3.8:1 |
| 9 | H | TsCl | DMAP | 0 | ACN | 3/50 | 1:1 |
| 10 | H | Boc₂O | 1-methyl-imidazole | 0 | ACN | 1/30 | 2:1 |
| 11 | H | MsCl | 1-methyl-imidazole | −40 | DME/ACN | 4/50 | 4.3:1 |
| 12 | H | MsCl | Pyridine | −40 | Pyridine | 6/10 | 5:1 |
| 13 | H | MstCl | 1-methyl-imidazole | −40 | DME/ACN | 12/50 | 4.3:1 |

### Example 4

### Classical Resolution of (±)-Cis-3-hydroxy-4-phenyl-azetidin-2-one

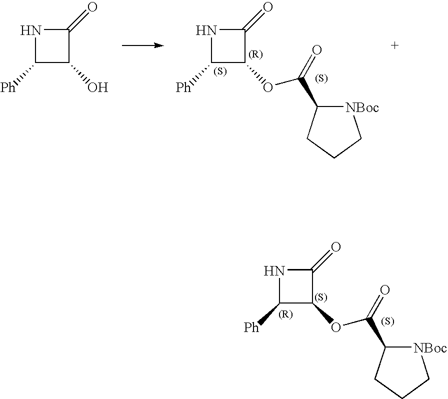

As an alternative to the above kinetic resolution, the diastereomeric mixture of the proline esters was separated via recrystallization from ethyl acetate. Subsequent hydrolysis of the proline esters separately would yield both enantiomers of the beta-lactam and recover the chiral amino acid. Thus, to a solution of N-methyl-imidazole (12 g, 150 mmol) in acetonitrile (80 mL) at 0° C. was added methanesulfonyl chloride (MsCl, 5.7 g, 50 mmol) and stirred for 15 minutes until the exothermic reaction temperature was stable at 0° C. To this solution was added N-Boc-L-Proline (11 g, 50 mmol) portion-wise and stirred at 0° C. for 30 minutes. Racemic (±)-cis-3-hydroxy-4-phenyl-azetidin-2-one (8.2 g, 50 mmol) was added portion-wise and the mixture was stirred at this temperature until TLC monitoring (3:1/ethyl acetate:hexanes) indicated complete conversion to the ester products after 1 h. The acetonitrile solvent was removed under rotary evaporation at 40° C. and the residue was taken up in ethyl acetate (500 mL), washed with water (100 mL), saturated aqueous sodium bicarbonate, brine, and dried over sodium sulfate. The drying agent was removed by vacuum filtration and the filtrate was concentrated to give 18 g of solid. A portion (7 g) of the mixture was taken up in 40° C. ethyl acetate (60 mL) and crystals (1.5 g) were formed at 40° C.; the crystals were collected and shown to be the desired 3R,4S-diastereomer of the (2S)-tert-butyl (3R,4S)-2-oxo-4-phenylazetidin-3-yl pyrrolidine-1,2-dicarboxylate. ¹H NMR (400 MHz, CDCl₃) δ (ppm): This diastereomer exists as a 1.7:1 (δ (ppm) 5.84:5.87) pair of diastereomers on the NMR timescale as typified by the characteristic chemical shift change of the starting material C3-carbinol proton from a multiplet at 5.12 ppm downfield to 5.8 ppm as a pair of doublet of doublets (J=4.68, 2.57 Hz) in the esterified product.

The filtrate was allowed to stand at ambient temperature for 5 h to give a second form of crystals (2.4 g) shown to be the 3S,4R-diastereomer of (2S)-tert-butyl (3S,4R)-2-oxo-4-phenylazetidin-3-yl pyrrolidine-1,2-dicarboxylate. ¹H NMR (400 MHz, CDCl₃) δ (ppm): This diastereomer exists as a 1:1.9 (δ(ppm) 5.90:5.94) pair of diastereomers on the NMR timescale as typified by the characteristic chemical shift change of the starting material C3-carbinol proton from a multiplet at 5.12 ppm downfield to 5.9 ppm as a pair of doublet of doublets (J=4.68, 2.57 Hz) in the esterified product.

Differences between of the classical thermodynamic controlled resolution and the kinetic resolution is that a stoichiometric amount of reagents are used and careful low temperature control is not critical. However, classical resolution requires one additional step of de-esterification of the diastereomeric ester to recover the desired C3-hydroxy substituted β-lactam.

### Example 5

### Optically active (+)-cis-3-trimethylsilyloxy-4-phenyl-azetidin-2-one

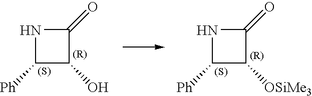

Optically active (+)-cis-3-hydroxy-4-phenyl-azetidin-2-one (3.4 g, 20.8 mmol) was dissolved in THF (30 mL) along with triethylamine (5.8 g, 57.4 mmol) and DMAP (76 mg, 0.62 mmol) at 0° C. Trimethylsilyl chloride (2.4 g, 22 mmol) was added dropwise and the mixture stirred for 30 min. TLC (3:1 ethyl acetate:heptane) showed complete conversion to the less polar product. The mixture was diluted with ethyl acetate (30 mL), washed with saturated aqueous sodium bicarbonate (15 ml), brine (15 ml), and dried over sodium sulfate (5 g). The sodium sulfate was filtered and the filtrate was concentrated and solvent exchanged with heptane (50 mL) to give a white powder. The powder was collected via vacuum filtration through a Buchner funnel and dried under vacuum (<1 mmHg) at ambient temperature to a constant weight of 3.45 g (72% yield). mp: 120 to 122° C., [α]²²₅₇₈=+81.9 (MeOH, 1.0), ¹H NMR (400 MHz, CDCl₃) δ (ppm): −0.08 (s, 9H), 4.79 (d, J=4.4 Hz, 1H), 5.09 (dd, J=4.4, 2.7 Hz, 1H), 6.16 (bm, 1H), 7.3 to 7.4 (m, 5H).

### Example 6

### Optically Active (+)-Cis-N-t-butoxycarbonyl-3-trimethylsilyloxy-4-phenyl-azetidin-2-one

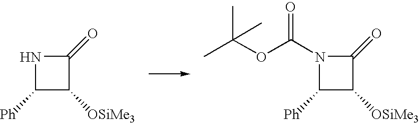

To a solution of optically active (+)-cis-3-trimethylsilyloxy-4-phenyl-azetidin-2-one (0.95 g, 4 mmol) in THF (10 mL) was added triethylamine (1.1 g, 5 mmol), DMAP (15 mg, 0.12 mmol) and di-t-butyldicarbonate (Boc₂O, 5.04 g, 5 mmol). The mixture was stirred at ambient temperature until the evolution of gas ceased and complete conversion to a less polar product was observed via TLC (2:1 ethyl acetate:heptane). The reaction mixture was diluted with heptane (20 mL) and filtered through a pad of silica gel (10 g) and concentrated in a 30° C. rotary evaporator until crystal formation occurred. The crystals were collected via vacuum filtration through a Buchner funnel, washed with cold heptane, and dried under vacuum (<1 mmHg) at ambient temperature to a constant weight of 0.87 g (65%). mp: 85 to 88° C., [α]²²₅₇₈=+106.9 (MeOH, 1.0), ¹H NMR (400 MHz, CDCl₃) δ (ppm): −0.07 (s, 9H), 1.38 (s, 9H), 5.01 (d, J=5.6 Hz, 1H), 5.06 (d, J=5.6 Hz, 1H), 7.26 to 7.38 (m, 5H).

### Example 7

### (+)-Cis-N-benzoyl-3-(2-methoxy-2-propoxy)-4-phenyl-azetidin-2-one from (+)-Cis-3-hydroxy-4-phenyl-azetidin-2-one

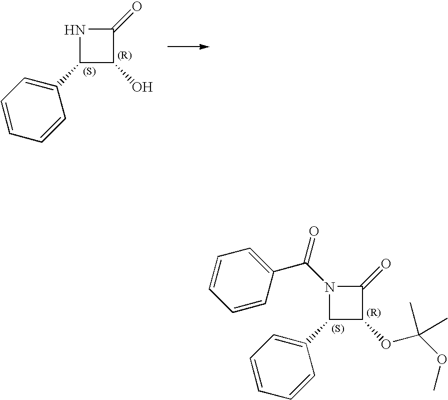

(+)-Cis-3-hydroxy-4-phenyl-azetidin-2-one (13.67 g, 83.8 mmol) was dissolved in anhydrous THF (275 mL) under nitrogen at a concentration of 20 mL/g, cooled to −15 to −10° C., and TsOH monohydrate (0.340 g, 1.8 mmol) was added. To the reaction at this temperature was added drop-wise 2-methoxypropene (6.49 g, 90 mmol). A sample of the reaction mixture was quenched with 5% TEA in ethyl acetate and the conversion to the intermediate was monitored by TLC (3:1 ethyl acetate:Heptane). Once the reaction was complete, triethylamine (25.5 g, 251 mmol) and DMAP (0.220 g, 1.8 mmol) were added. Benzoyl chloride (12.95 g, 92.18 mmol) was added to the reaction mixture before warming to ambient temperature and stirred until the conversion to (+)-cis-N-benzoyl-3-(2-methoxy-2-propoxy)-4-phenyl-azetidin-2-one was complete (3 to 5 h). The mixture was diluted with heptane equal in volume to the THF. The solid salt was filtered off and the mixture was washed with water, saturated aqueous sodium bicarbonate and brine. The organic phase was filtered through silica gel and the filtrate was concentrated until crystals formed. The solid was collected by vacuum filtration and washed with heptane:triethylamine (95:5) as a white solid 21.0 g, 61.9 mmol, 74% yield). Mp:98 to 100° C. ¹H NMR (400 MHz, CDCl₃) δ (ppm): 0.99 (s, 3H), 1.54 (s, 3H), 3.15 (s, 3H), 5.27 (d, J=6.3 Hz, 1H), 5.41 (d, J=6.3 Hz, 1H), 7.30 to 7.43 (m, 5H), 7.47 (t, J=7.54 Hz, 2H), 7.59 (m, J=7.54 Hz, 1H)), 8.02 (m, J=7.54 Hz, 2H).

What is claimed is:

1. A process for the resolution of an enantiomeric mixture of first and second C3-hydroxy substituted α-lactam enantiomers comprising
(a) treating the enantiomeric mixture with an optically active proline acylating agent in the presence of an amine to form a product mixture, the product mixture containing first and second C3-ester substituted β-lactam diastereomers formed by reaction of the first and second C3-hydroxy substituted β-lactam enantiomers, respectively, with the optically active proline acylating agent, the product mixture optionally also containing unreacted second C3-hydroxy β-lactam enantiomer, and
(b) separating the first C3-ester substituted β-lactam diastereomer from the unreacted second C3-hydroxy β-lactam enantiomer or the second C3-hydroxy substituted β-lactam diastereomer.

2. The process of claim 1 wherein substantially all of the first enantiomer in the enantiomeric mixture is converted to the first C3-ester substituted β-lactam diastereomer but substantially all of the second enantiomer in the enantiomeric mixture remains unreacted in the product mixture.

3. The process of claim 1 wherein substantially all of the first and second enantiomers in the enantiomeric mixture are converted to the first and second C3-ester substituted β-lactams in the product mixture.

4. The process of claim 1 wherein the optically active proline acylating agent is prepared by treating an optically active proline or proline derivative with an acid acylating agent and an amine.

5. The process of claim 1 wherein either the unreacted second enantiomer or the second diastereomer are separated from the first diastereomer by crystallization.

6. The process of claim 1 wherein the optically active proline acylating agent is an acid halide, anhydride, or mixed anhydride of N-t-butoxycarbonyl-L-proline or N-carbobenzyloxy-L-proline.

7. The process of claim 4 wherein the optically active proline acylating agent is prepared by treating N-t-butoxycarbonyl-L-proline or N-carbobenzyloxy-L-proline with an acid acylating agent and an amine.

8. The process of claim 4 wherein the acid acylating agent is p-toluenesulfonyl chloride (TsCl), methanesulfonyl chloride (MsCl), oxalic acid chloride, di-t-butyl dicarbonate (Boc₂O), dicyclohexylcarbodiimide (DCC), alkyl chloroformate, 2-chloro-1,3,5-trinitrobenzene, polyphosphate ester, chlorosulfonyl isocyanate, Ph₃P—CCl₄ or combinations thereof.

9. The process of claim 1 wherein the amine is an aromatic amine wherein the aromatic amine is a substituted or unsubstituted pyridine, a substituted or unsubstituted imidazole, or combinations thereof.

10. The process of claim 9 wherein the aromatic amine is pyridine N,N′-dimethylaminopyridine (DMAP), imidazole, 1-methylimidazole, 1,2-dimethylimidazole, benzimidazole, N,N′-carbonyldiimidazole, or combinations thereof.

11. The process of claim 1 wherein the β-lactams in the enantiomeric mixture have the Formulae cis-1 and cis-2 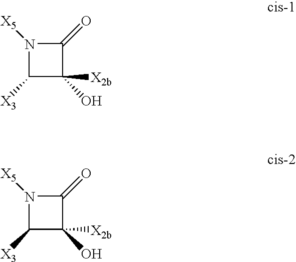
wherein
X_{2b} is hydrogen, alkyl, alkenyl, alkynyl, aryl, heterocyclo, or —SX₇;
X₃ is alkyl, alkenyl, alkynyl, aryl, acyloxy, alkoxy, acyl or heterocyclo or together with X₅ and the carbon and nitrogen to which they are attached form heterocyclo; and
X₅ is hydrogen, hydrocarbyl, substituted hydrocarbyl, —COX₁₀, —COOX₁₀, —CONX₈X₁₀, —SiR₅₁R₅₂R₅₃, or together with X₃ and the nitrogen and carbon to which they are attached form heterocyclo;
X₇ is hydrogen, hydrocarbyl, substituted hydrocarbyl, or heterocyclo;
X₈ is hydrogen, hydrocarbyl, substituted hydrocarbyl, or heterocyclo;
X₁₀ is hydrocarbyl, substituted hydrocarbyl, or heterocyclo; and
R₅₁, R₅₂, and R₅₃ are independently alkyl, aryl or aralkyl.

12. The process of claim 11 wherein X_{2b} is hydrogen.

13. The process of claim 11 wherein X₃ is isobutenyl, cyclopropyl, cyclopentyl, phenyl, thienyl, furyl, or pyridyl.

14. The process of claim 11 wherein X₅ is hydrogen, —COX₁₀ wherein X₁₀ is phenyl, —COOX₁₀ wherein X₁₀ is n-propyl, isopropyl, n-butyl, isobutyl or tert-butyl, or —SiR₅₁R₅₂R₅₃ wherein R₅₁, R₅₂, and R₅₃ are methyl.

15. A β-lactam compound having the structure of Formula 4 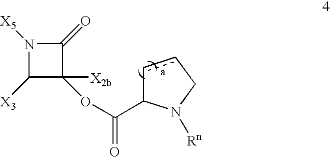
wherein
a is 1 or 2 whereby the heterocyclo ring is proline or homoproline;
the dashed line denotes an optional double bond between the C3 and C4 ring carbon atoms;
Rⁿ is a nitrogen protecting group;
X_{2b} is hydrogen, alkyl, alkenyl, alkynyl, aryl, heterocyclo, or —SX₇;
X₃ is alkyl, alkenyl, alkynyl, aryl, acyloxy, alkoxy, acyl or heterocyclo or together with X₅ and the carbon and nitrogen to which they are attached form 10 heterocyclo;
X₅ is hydrogen, hydrocarbyl, substituted hydrocarbyl, —COX₁₀, —COOX₁₀, —CONX₈X₁₀, —SiR₅₁R₅₂R₅₃, or together with X₃ and the nitrogen and carbon to which they are attached form heterocyclo;
X₇ is hydrogen, hydrocarbyl, substituted hydrocarbyl, or heterocyclo;
X₈ is hydrogen, hydrocarbyl, substituted hydrocarbyl, or heterocyclo;
X₁₀ is hydrocarbyl, substituted hydrocarbyl, or heterocyclo; and
R₅₁, R₅₂, and R₅₃ are independently alkyl, aryl or aralkyl.

16. The β-lactam compound of claim 15 wherein Rₙ is t-butoxycarbonyl or carbobenzyloxy.

17. The β-lactam compound of claim 15 wherein X_{2b} is hydrogen.

18. The β-lactam compound of claim 15 wherein X₃ is isobutenyl, cyclopropyl, cyclopentyl, phenyl, thienyl, furyl, or pyridyl.

19. The β-lactam compound of claim 15 wherein X₅ is hydrogen, —COX₁₀ wherein X₁₀ is phenyl, —COOX₁₀ wherein X₁₀ is n-propyl, isopropyl, n-butyl, isobutyl or tert-butyl, or —SiR₅₁R₅₂R₅₃ wherein R₅₁, R₅₂, and R₅₃ are methyl.

20. The β-lactam compound of claim 15 wherein a is 1.
